(19) United States
(12) Patent Application Publication
(10) Pub. No.: US 2006/0110756 A1
(43) Pub. Date: May 25, 2006

(54) Large-scale parallelized DNA sequencing

(76) Inventors: Tom Tang, San Jose, CA (US); Radoje Drmanac, Los Altos Hills, CA (US)

Correspondence Address: BELL & ASSOCIATES, 416 FUNSTON ST., SUITE 100, SAN FRANCISCO, CA, 94118, US

(21) Appl. No.: 11/258,775

(22) Filed: Oct. 25, 2005

Related U.S. Application Data
(60) Provisional application No. 60/621,849, filed on Oct. 25, 2004.

(51) Int. Cl. C12Q 1/68; C12M 1/34

(52) U.S. Cl. 435/6; 435/287.2

(57) ABSTRACT

We provide a DNA sequencing method and a sequencing system where large numbers of sequence reads can be obtained in parallel by running traditional electrophoresis in a special format. Parallelization is obtained either through a 3-dimensional gel-cube or through bundled capillary tubes including fiber-optic tubes or other types of micro channels in a bundle or matrix format. Various ways of capturing sequence traces are provided. We also provide two distinct methods for preparing genomic DNA/cDNA fragments: one through universal primer site anchoring and amplification of single molecules, and the other through micro-array/bead oligomer extension and dye-terminator incorporation using target sequence specific primers. The invention can perform large-scale genomic sequencing including sequencing a complete human genome in one or a few runs.

The present application claims priority to U.S. Provisional Patent Application Ser. No. 60/621,849 entitled “Large-scale Parallelized DNA Sequencing”, filed Oct. 25, 2004, which is herein incorporated by reference in its entirety for all purposes.

### BACKGROUND TO THE INVENTION

Methods of determining the sequence of nucleic acids are some of the most important tools in the field of molecular biology. Since the development of the first methods of DNA sequencing in the 1970s, sequencing methods have progressed to the point where a majority of the operations are now automated, thus making possible the large scale sequencing of whole genomes, including the human genome. There are two broad classes of DNA sequencing methodologies: (1) the chemical degradation or Maxam & Gilbert method and (2) the enzymatic or dideoxy chain termination method (also known as the Sanger method), of which the latter is the more commonly used and is suitable for automation.

Of particular interest in DNA sequencing are methods of automated sequencing, in which fluorescent labels are employed to label the size separated fragments or primer extension products of the enzymatic method. In general, three different methods have been used for automated DNA sequencing. In the first method, the DNA fragments are labeled with one fluorophore and then run in adjacent sequencing lanes, one lane for each base. See Ansorge et al., Nucleic Acids Res. (1987) 15: 4593-4602. In the second method, the DNA fragments are labeled with oligonucleotide primers tagged with four fluorophores and all of the fragments are run in one lane. See Smith et al., Nature (1986) 321: 674-679. In the third method, each of the different chain terminating dideoxynucleotides is labeled with a different fluorophore and all of the fragments are run in one lane. See Prober et al., Science (1987) 238: 336-341.

The first method has the potential problems of lane-to-lane variations as well as a low throughput. The second and third methods require that the four dyes be well excited by one laser source, and that they have distinctly different emission spectra. Otherwise, multiple lasers have to be used, increasing the complexity and the cost of the detection instrument. With the development of Energy Transfer primers that offer strong fluorescent signals upon excitation at a common wavelength, the second method produces robust sequencing data in currently commercial available sequencers. However, even with the use of Energy Transfer primers, the second method is not entirely satisfactory. In the second method, all of the false terminated or false stop fragments are detected resulting in high backgrounds. Furthermore, with the second method it is difficult to obtain accurate sequences for DNA templates with long repetitive sequences. See Robbins et al., Biotechniques (1996) 20: 862-868.

The third method has the advantage of only detecting DNA fragments incorporated with a terminator. Therefore, backgrounds caused by the detection of false stops are not detected. However, the fluorescence signals offered by the dye-labeled terminators are not very bright and it is still tedious to completely clear up the excess of dye-terminators even with AmpliTaq DNA Polymerase (FS enzyme). Furthermore, non-sequencing fragments are detected, which contributes to background signal. See Applied Biosystems Model 373 A DNA Sequencing System User Bulletin, November 17, P3, August 1990.

Current automated DNA sequencing methods primarily uses capillary gel electrophoresis. Each capillary (usually between 1 and 96) is loaded with prepared sample from a tube or a multi-well plate. Single file array of capillaries or etched micro-channels is read toward the end or at the exit during the electrophoresis time. The system has two main limitations: cost and time in sample preparation and a limited throughput of parallel reactions.

Thus, there is a need for the development of improved methodology that is capable of providing for faster and significantly less-costly methods and tools for sequencing DNA.

### SUMMARY OF THE INVENTION

The invention provides DNA sequencing instruments, systems, kits, methods, and processes for sequencing more than 1000 single polynucleotides simultaneously. In a preferred embodiment the invention provides the sequence of a genome with at least 2× coverage. In a more preferred embodiment, the invention provides the sequence of a genome with at least 4× coverage. In a still more preferred embodiment, the invention provides the sequence of a genome with at least 8× coverage. In a most preferred embodiment, the invention provides the sequence of a genome with at least 16× coverage.

In a first embodiment the invention provides a process for sequencing DNA, the process comprising: parallelized preparing of more than 1000, 10,000, 100,000, or 1,000,000 DNA sequencing reactions using three or four dyes, labels or tags corresponding to specific DNA bases; parallelized loading of prepared DNA fragments on a separation matrix with corresponding capacity; running electrophoresis separation of DNA fragments and illuminating and detecting three or four dyes, labels or tags in time points for each separation element at specific location close to the end, inside or outside, of separation medium; and determining base sequence from the time profile of intensities of three or four dyes, labels or tags in more than 1000, 10,000, 100,000, or 1,000,000 DNA samples run in parallel.

In a second embodiment, the invention provides a process for sequencing DNA, the process comprising: parallelized preparing of more than 1000, 10,000, 100,000, or 1,000,000 DNA sequencing reactions using target sequence specific primers attached to beads or to an array support; parallelized loading of beads or labeled DNA fragment to gel cube or matrix of sequencing capillaries by gravitational, capillary or electric forces; running electrophoretic separation of DNA fragments and illuminating and detecting four dyes in time points at specific location close to the end, inside or outside of separation medium; and determine base sequence from the time profile of intensities of four colors in more than 1000, 10,000, 100,000, or 1,000,000 DNA samples run in parallel.

In a third embodiment the invention provides a process for sequencing DNA, the process comprising: parallelized DNA amplification from more than 1000, 10,000, 100,000, or 1,000,000 single molecules using universal primers in a matrix having a corresponding number of microstructures loaded by capillary forces; parallelized sequencing reaction with four dye terminators in the same matrix of microstrucutres that may be loaded with beads with sequencing primer; parallelized loading of samples from matrix of microstructure to matrix of sequencing capillaries by capillary or electric forces; runing electrophoretic separation of DNA fragments and illuminating and detecting four flourophores in time points at specific location close to the end, inside or outside of capillaries; and determine base sequence from the time profile of intensities of four colors in more than 1000, 10,000, 100,000, or 1,000,000 samples run in parallel.

In a fourth embodiment the invention provides a system for parallelized amplification of polynucleotides and incorporation of dye-terminator into the polynucleotides consisting of a matrix of more than 1000, 10,000, 100,000 or 1,000,000 micro-wells or micro channels with porous bottom, and micro-beads of corresponding size cable of attaching or with attached sequencing primers.

In an alternative embodiment, the system for parallelized amplification and dye-terminator incorporation consists of a matrix of more than 1000, 10,000, 100,000 or 1,000,000 micro-wells or micro-channels with porous bottom and walls capable of attaching or with attached one or both amplification primers, and micro-beads of corresponding size cable of attaching or with attached sequencing primers.

In another alternative embodiment, the system for parallelized amplification and dye-terminator incorporation consists of a matrix of more than 1000, 10,000, 100,000 or 1,000,000 micro-wells or micro channels with porous bottom, and two sets of micro-beads of corresponding size, one cable of attaching or with attached amplification primers, and one cable of attaching or with attached sequencing primers.

In a fifth embodiment the invention comprises an instrument for sequencing DNA comprising a gel-cube or a matrix or bundle of capillaries or fibers or channels with more than 1000, 10,000, 100,000 or 1,000,000 elements.

In an alternative embodiment, the DNA sequencing instrument comprises a gel-cube or a matrix or bundle of capillaries or fibers or channels with more than 1000, 10,000, 100,000 or 1,000,000 elements and a compatible kit for parallel preparation and loading of comparable number of DNA samples based on amplification of single molecule in microstructures and/or on beads, or using rolling circle amplification, or sorting natural or amplified copies of DNA fragments from a mix of fragments using target sequence specific primers attached to array surface or beads.

In another alternative embodiment, the DNA sequencing instrument comprises a matrix or bundle of capillaries or fibers or channels with more than 1000, 10,000, 100,000 or 1,000,000 elements, where the elements are bent at the exit end and illuminated at an angle that reflects light outside of sequencing capillaries. In another alterative, the exit end of the capillary can have a prismatic shape and the light be refracted by the prism. In a further alterative, the base of the medium, such as the gel-box of fiber matrix, can comprise a plurality of tilted reflecting surfaces comprising a reflective compound.

In a still further alterative embodiment, the DNA sequencing instrument comprises a matrix or bundle of capillaries or fibers or channels with more than 1000, 10,000, 100,000 or 1,000,000 elements, and a mechanism for consecutive depositing of exiting labeled DNA on a substrate and a subsystem for imaging printed arrays of DNA. In one embodiment, the mechanism can comprise means for depositing the DNA upon a substrate the means selected from the group consisting of a liquid sprayer, an ink-let printer or the like, a charged plate for donating ions to a fluid, and a bubble-jet electrode. In one embodiment the subsystem can comprise means for imaging a printed DNA array, the means selected from the group consisting of a photon detector, an electron detector, and a confocal fluorescence scanner.

In a sixth embodiment the invention provides a system for sequencing DNA comprising a DNA preparation and loading matrix of microstructures that correspond to a DNA separation/sequencing matrix, each with more than 1000, 10,000, 100,000 or 1,000,000 elements.

In an alterative embodiment, the DNA sequencing system comprises a DNA preparation and loading matrix of microstructures that correspond to a DNA separation/sequencing matrix, each with more than 1000, 10,000, 100,000 or 1,000,000 elements, where the elements are bent at the exit end and illuminated at an angle that reflects light outside of sequencing capillaries.

In another alternative embodiment, the DNA sequencing system comprises a DNA preparation and loading matrix of microstructures that correspond to a DNA separation/sequencing matrix, each with more than 1000, 10,000, 100,000 or 1,000,000 elements, and a mechanism for consecutive depositing of exiting labeled DNA on a substrate and a subsystem for imaging printed arrays of DNA.

In another embodiment the DNA sequencing instrument comprises a gel-cube capable of running more than 1000, 10,000, 100,000 or 1,000,000 elements.

In another embodiment the DNA sequencing system comprises a DNA preparation and loading matrix of microstructures and gel cube capable of simultaneous loading and running more than 1000, 10,000, 100,000 or 1,000,000 sequencing reactions.

In a seventh embodiment the invention provides a reaction microarray or a reaction micromatrix for hybridizing DNA and for sequencing DNA, the reaction microrray or micromatrix comprising spotted primers having a density of 1,000, 1,001-10,000, 10,001-100,000, 100,001-1,000,000, 1,000,001-10,000,000 spots per microarray or micromatrix, where each spot comprises a specific primer sequence having a length of 10-20 bp, 21-30 bp, 31-50 bp, 50-100 bp, the primer sequence providing an anchor that hybridizes with a mixture of DNA fragments to be sequenced; the spotted primers further comprising an anchor fragment that can be released by heat or chemical reagents; and wherein under hybridization conditions the spotted primers hybridize to DNA fragments that contain the complimentary sequence to the last portion of the sequence; wherein hybridizations having miss-matches are removed using heat or physical means that results in the hybridized fragments having greater purity or identity; wherein the hybridized fragments are used as a template in a sequencing reaction wherein the anchored primers are extended by DNA polymerase, nucleotides, and dye-terminators are randomly incorporated into certain portions of primers; wherein the hybridized DNA fragments are decoupled from the anchored strand using heat or physical means and the microarray or micromatrix is washed to remove the unanchored DNAs; wherein the anchored DNA is released from the surface of microarray or micromatrix using enzymic or physical means; and wherein the released DNAs are passed through microfibers or gel-cubes for sequencing.

In an eighth embodiment the invention provides a process for parallel preparation of a sequencing reaction using sequence specific primers, the process comprising the steps of: i) providing a plurality of attached releasable primers selected from the group consisting of 10-1,000, 1,001-10,000, 10,001-100,000, 100,001-1,000,000, and 1,000,000-10,000,000; ii) contacting and anchoring each primer with a substrate to create at least one spot comprising the primer, wherein the substrate is selected from the group consisting of a microarray plate, a bead, and a micro-structure, wherein each spot comprises a primer sequence having length selected from the group consisting of 10-20 bp, 21-30 bp, 31-50 bp, and 50-100 bp, and wherein the primer is designed for a genome or a set of genomes; iii) hybridizing a mixture of DNA fragments to be sequenced isolated from the genome to the complementary primers under stringent conditions; iv) optionally purifying the hybridized DNA fragment having miss-matches using heat or physical means v) sequencing DNA fragments using nucleotides and dye-terminators and the hybridized fragments as a template whereby the anchored primers are extended by DNA polymerase and the dye-terminators are incorporated in the growing polynucleotide chain at random positions; vi) optionally decoupling the DNA fragments fro the achored primer strand using heat or physical means; washing the substrate to remove free DNA; vii) releasing the anchored DNA from the surface of the substrate via enzymes or physical means; and viii) passing the released DNA through microfibers or gel-cubes for sequencing.

In a ninth embodiment the invention provides a reaction substrate having a plurality of surfaces comprising a composition suitable for sequencing polynucleotides, re-sequencing polynucleotides, genotyping, and SNP discovery, the substrate further comprising a plurality of primers anchored to the substrate and wherein each primer sequence is complementary to a specific polynucleotide sequence in a polynucleotide or genome of interest and wherein the primer further comprises a releasable anchor fragment, wherein the anchor fragment is released using means selected from the group consisting of heat and by chemical reagents, such as, but not limited to, enzymes and catalysts, and wherein the released polynucleotide is passed through a medium selected from the group consisting of a microfiber and a gel-cube. In one embodiment the reaction substrate is selected from the group consisting of a microarray, a micromatrix, a microarray plate, a plurality of beads, and a micro-structure. In another embodiment the primers are at a density selected from the group consisting of 1,000, 1,001-10,000, 10,001-100,000, 100,001-1,000,000, and 1,000,001-10,000,000 primers per substrate. In a further embodiment the primers are of length selected from the group consisting of between about 10-20 bp, about 21-30 bp, about 31-50 bp, about 50-100 bp, about 101-200 bp, and about 201-400 bp. In a still further embodiment the primers are selected from the group consisting of random primers and primers having known polynucleotide sequence.

The invention also provides a method for sequencing DNA fragments using the reaction substrate as disclosed herein, the method comprising the steps of: i) providing the reaction substrate disclosed herein; ii) providing DNA fragments of interest; iii) hybridizing under stringent conditions DNA fragments that contain the complimentary sequence to the portion of the primer that is releasable; iv) optionally removing DNA fragments having miss-matches to the primers resulting in the hybridized DNA fragments having greater purity, wherein removing the DNA fragments is performed using means selected from the group consisting of heat and physical means; v) adding DNA polymerase, nucleotides, and dye-terminators to the reaction substrate; vi) incubating the DNA polymerase, nucleotides, and dye-terminators with the primers and hybridized DNA fragments to extend the primers complementary to the DNA fragments using the DNA fragments as a template in a sequencing reaction wherein the primers are extended to form a strand and whereby the dye-terminators are randomly incorporated into certain portions of primers to create an anchored DNA; vii) decoupling the hybridized DNA fragments from the anchored strand using means selected from the group consisting of heat and physical means, the means being selected from the group consisting of low stringency wash at 50° C. and a high stringency wash at 42° C.; viii) washing the substrate thereby removing the decoupled DNA; ix) releasing the anchored DNA from the surface of the substrate using enzymic or physical means; and x) passing the released DNA through a medium; sequencing the DNA in the medium using three-dimensional imaging, the medium comprising three-dimensional microstructures selected from the group consisting of bundles of capillary fibers, a gel-cube, and a mesh.

In a tenth embodiment the invention provides an oligomer extension and sequencing system, device, kit, and a process comprising of all or some of the following steps or elements:
1) spotted or in situ made oligomers fixed at one end on a solid surface or porous matrix or channel micro structures (similar to described above for target DNA amplification) or at entry portions of separation capillaries, or support in form of beads or other discrete physical particles or molecular structures with specific linkers that can be released from the support surface;
2) the oligomers designed to hybridize specifically to target sequences (produced by fragmentation and optional amplification of the mix of entire genome, chromosome, clone, or mixtures of clones or mixture of isolated genomic segments and mixture of primers used for preparation of targeted segments may contain the same primers used in step 1, providing that complementary DNA is produced), that contains the complimentary segment to the oligomer, and such hybridization occurs at controlled temperature (including cycling between discriminative and higher than discriminative temperature) and hybridization and mixing condition and reaction time such that unspecific hybridization is reduced to an acceptable level;
3) oligomer extension cycles during which deoxynucleotides (normal deoxynucleotides A, T, G, C and dye terminators fixed with fixed ratios) can be added onto the oligomer using the hybridized sequence as a template and the enzyme of DNA polymerase; cycle sequencing reaction may be used if there is more attached primers than hybridized templates;
4) optional removal of DNA template using high temperature and other denaturing conditions or exonuclease treatment, and optionally washing away of DNA fragments;
5) an optional step of removing those extended sequences without the dye-terminator at the end by specific enzymes; the removing step is to get a cleaner electrophoresis and higher quality;
6) releasing the extended oligomers with dye-terminator at the end from the support surface by the specific enzyme or chemical that can cut at the linker site followed by simultaneous and a spot or a bead to a gel spot or a capillary loading of denatured labeled fragments using capillary or electric forces;
   wherein the support surface is selected from the group consisting of glass, plastic, and metal surface seen in typical microarray settings, and wherein the surface of the microbeads is selected from the group consisting of plastic, metal, magnetic, or any other materials; and the matrix is selected from the group consisting of any polymer appropriate for fixing DNA sequences.

### BRIEF DESCRIPTION OF THE DRAWINGS

FIG. 1 illustrates the gel-cube (A) and capillary fiber matrix (B) in one aspect of the invention.

FIG. 2 illustrates an alternative embodiment of the invention showing arrays of gel-cubes or fibers.

FIG. 3 illustrates three different methods of using devices that may be used to read and determine the nucleotide sequence of the DNA.

FIG. 4 illustrates an exemplary embodiment if the invention showing how fibers emerging from a three-dimensional cube-shaped apparatus may be realigned into a one-dimensional array for scanning.

FIG. 5 illustrates three different exemplary ways and means for reflecting excitation photons.

FIG. 6 illustrates four exemplary DNA fragments that can be used with the invention.

FIG. 7 illustrates a cartoon showing the random distribution of the single copy genomic DNA (open circles) that are the substrate for the amplification process.

FIG. 8 illustrates an exemplary protocol for selecting oligomers that results in a 2× coverage of the double-stranded genomic region following amplification.

FIG. 9 illustrates a method of generating dye-terminator ended polynucleotides from random fragments of genomic DNA.

FIG. 10 illustrates an exemplary capillary array wherein beads comprising DNA fragments are placed upon the end of a capillary; enzymes degrade the bead thereby sequentially releasing the DNA fragments.

### DETAILED DESCRIPTION OF THE INVENTION

The invention provides DNA sequencing instruments, systems, kits, methods, and processes for sequencing more than 1000 distinct polynucleotides simultaneously. The invention further contemplates that more than one million such polynucleotides can be sequenced simultaneously. The invention also contemplates sequencing polynucleotides in three dimensions (i.e. a plurality of labeled polynucleotides can be migrated through a single microfiber) using the systems and methods disclosed herein.

We proposed methods, devices, and instruments that dramatically simplify sample preparation and loading, electrophoresis, and reading of very large number of sequencing reactions in parallel. The new methods dramatically increase sequencing capacity. New instruments are capable of performing tens of thousands or hundreds of thousands of parallel sequencing reactions.

Our method is based on employing proven gel-electrophoresis or other separation process run on a new highly parallel system and combined with highly parallel amplification or with microarray technology. This method has the potential of sequencing the complete human genome with a single read, it can report all the SNPs and the genotypes of each haploid chromosome, it can be used for scientific research, drug discovery and development, and it can be used for genetic testing and diagnostics in humans (including screening for preventive and predictive personalized medicine), animals, plants, food, water, air or any environmental samples. Compared with current sequencing methods explored by others such as sequencing by in situ synthesis or pyro-sequencing, the disclosed method is simple and direct, and with a longer read length. Many components used with the invention, such as microarrays with spotted or synthesized oligomers, in situ amplification of random sequences, gel-cubes, and capillary arrays, are all available in various formats.

A number of different reaction substrates are contemplated, including microarray surfaces; microarray plates; a micromatrix having a three-dimensional surface comprising compounds such as, but not limited to, polymeric compounds, gels, foam compounds, high-viscosity fluids, or the like, having pores or the like, the pores having dimensions suitable for allowing through-passage of small molecules but reducing or preventing the diffusion of macromolecules, such as polynucleotides or the like, but that when the substrate is subjected to an electric current or electromagnetic radiation allows the macromolecule to move through the substrate; a collection of beads; a micro-structure; or the like.

Similar to the improvements in semiconductor density in the microelectronic industry, we can improve on the current technology. For example, by improving or optimizing the capturing of signals from the dye terminator, we can reduce the number of oligomers needed to be spotted at each site, and therefore increase the density of microarray, or we can extend the read length with the same oligomer density. The eventual bottleneck, of course, is the detection limit: how many molecules with the same dye-terminators at each fixed length are required for detection. There is probably a limit on the improvement in that we cannot increase below the single-molecule level. In that sense, the spotted oligomers at each spot or the number of template molecules produced by single molecule amplification multiplied by number of extension cycles in dye-terminators incorporation have to be >1,000 if we intend to generate read length >1,000. On the other hand, our calculations show that even with typical resolutions and yields that can be efficiently achieved today we can to generate the whole genomic sequence in a single experiment.

We provide examples where the technology disclosed herein can be applied to sequence the complete human genome, or the like, with a single or small number of instrument runs using DNA from newborn babies, patient samples, tumor tissues, or the like. Other applications of our technology are obvious, and we need not provide details here. These applications include, but are not limited to:
Sequencing individual eukaryotic chromosomes
Sequencing BACs or mixtures of BACs
Sequencing mixtures of genomic segments
Sequencing bacterial genomes (including the Archaea)
Sequencing yeast genomes
Sequencing plant genomes
Sequencing plastid genomes
Sequencing mitochondrial genomes
Sequencing the partial or complete cDNA/mRNA collection of expressed genes from individual or pooled mixtures of cDNA libraries.

I. Highly Parallelized Electrophoresis-Based DNA Sequencing

1. DNA Sample Preparation

A DNA fragment is a nucleotide segment that we would like to know the sequence. A prepared DNA sample is a mixture of subfragments of the DNA fragment with varying length, with dye-terminator placed at the 3′ end, one for each base (A, G, C, and T). The 3′ dye-terminators are capable of emission different color of lights when excited by a photon beam having a certain wavelength. We assume at each DNA sample is well-mixed with DNA subfragments of the nucleotide sequences. We may need about 100 molecules (possibly as little as one or a few) at each fragment length in order to generate a detectable signal for a detector. Thus, to sequence a nucleotide of 1,000 bp in length, we may need 100,000 molecules in the DNA sample. The concepts mentioned here are typical in today's sequencing devices.

In section III, two different scenarios are described where the DNA samples qualifying the above criteria can be prepared. In this section, it is assumed that such DNA samples are already available.

2. Gel-Cube Device

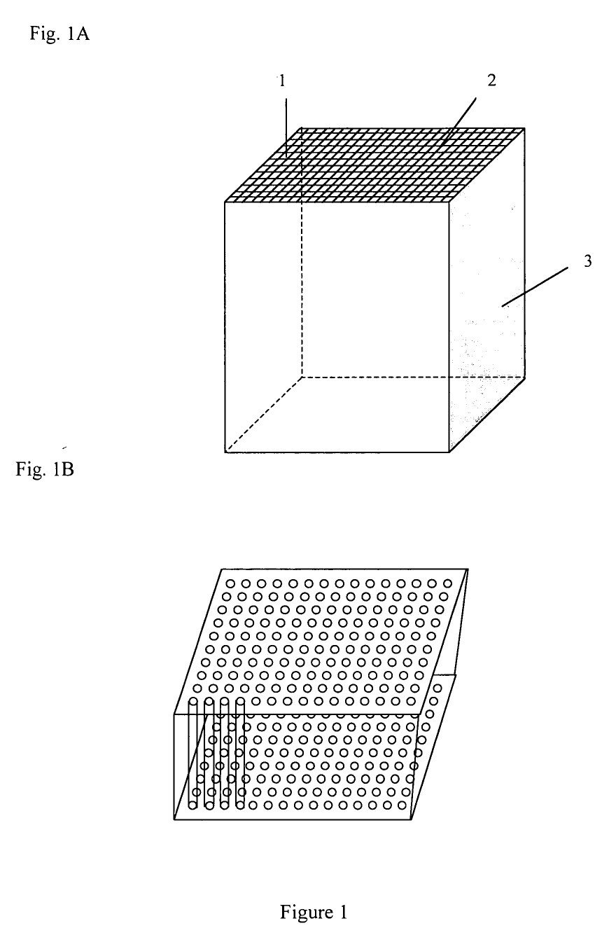

A gel block of a certain length, width, and height (for example, 1 cm*1 cm on the top, and 10 cm in height) is formed and is bounded within a solid container that is made of glass, metal, or plastic, or any other material. Those components combined together form a gel-cube (FIG. 1A).

We start with allocating a certain amount of DNA samples (for example, 1,000×1,000 DNA samples) into the gel-cube (evenly distributed or randomly distributed). For example, the even distribution of DNA samples can be from needle injections into the gel, with the DNA samples prepared outside. The randomly distributed DNA samples can be from an in situ amplification process (such as, but not limited to PCR, RT-PCR, using a DNA polymerase or fragments thereof, using a synthetic polymerase, chemical synthesis, or the like). The amount of DNA fragments allocated depends on the detection apparatus, and can be varying from numbers given here. For example, in a typical injection from needle head will contain from about 10⁵ to about 10⁸ number of sample molecules. A typical amplification yield is in the range of between about 10⁶-fold to about 10⁸-fold as well.

One technique to generate high quality sequence reads is to provide boundaries within the gel-cube. One way is to have many very thin physical layers (can be plastic, metal, etc.) within the gel, or even a vertical mesh. The layers should go vertical against the gel-cube. It may separate the gel-cube into many thin layers, or into many small vertical grids. This is to guarantee as the samples travel down the gel, they do not go astray or become entangled with each other, and also make the tracking of the trace easier.

3. Fiber Matrix

In one embodiment optic or capillary fibers or channels (fibers from here on) may be used to guide the samples (FIG. 1B). In this way, a fiber matrix, with thousands to millions of fibers tightly or loosely bundled together, is placed beneath and in contact with the prepared DNA samples, or samples are prepared on top of or inside of fibers. The DNA samples will run down only through individual fibers. Fibers may be of various material composition (various type of glass, plastic, polymer, or metal) and surface coating and optical properties. Fibers of different internal and external diameter can be used from a few microns (such as from between 1-10 microns) or from about 10-30 microns, or up to about 100 microns of internal diameter. Similarly, the external diameter of the fibers can be from between about 1-10 microns, about 10-30 microns, and up to more than about 100 microns. In addition, the center to center distance of two fibers can be from about 3-5 microns, about 5-10 microns, about 10-30 microns, and from between 30 to about 200 microns. For example, a square matrix with one million capillaries having center to center distance of 10 microns will have dimensions of about 1 cm×1 cm. The same size bindle with capillaries 100 microns center to center will have 10,000 capillaries and capacity of about 10 megabases (Mbp) per run. Many arrangements and sizes are possible for different applications. The capillary matrix may be reusable or disposable.

4. Using Arrays of Cubes or Arrays of Capillary Matrices

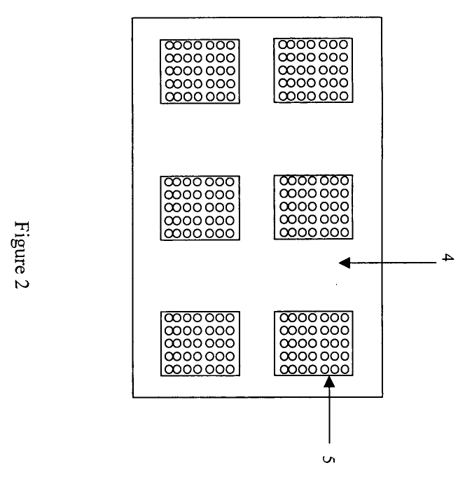

In one embodiment, an array of X by Y unit gel-cubes or capillary matrices is used to add additional flexibility and efficiency of the instrument (FIG. 2). An array may have total of 2 to 384 or more units. Some specific number of units may be 4, 8, 12, 16, 24, 32, 48, 96, 192, or 384. The array may match center-to-center dimensions of standard 96, 384, or 1536 well-plates. Each unit array can have a capacity for more than about 50, or 100, or 1000, or 10,000, or 100,000, or 1,000,000 reactions. The space between unit arrays may be of different material or be open and used for temperature regulation or flow of electrophoretic or other medium. Electrophoretic buffer and/or power control and/or illumination/detection may be isolated for each unit. All units may have the same or different gel-cube composition, separation medium composition, or capillary size or arrangements. Dimensions and arrangements of array of matrices of microstructures or multi-well plate that may be used for sample amplification and preparation have to match array of matrices of sequencing capillaries. The loading of unit arrays may be one at a time, multiple at a time or all simultaneously. The process may be integrated or robotized using multi-channel pipetting tools or capillary bundles. Such microfluidic applications and devices are well known to those in the art.

II. Imaging of the Running Samples

As electrical power is applied to the gel-cube from both sides, the dye-terminator labeled DNA fragments within each DNA sample will migrate downwards through the length of the gel with varying speed depending on their respective molecular weight. The task now is to capture the identity of each fragment as the fragment passes through a fixed imaging layer within or outside the gel-cube. The imaging layer is a 2-dimensional layer that is parallel to the top surface of the gel-cube.

1. Focusing at Distinct Layers

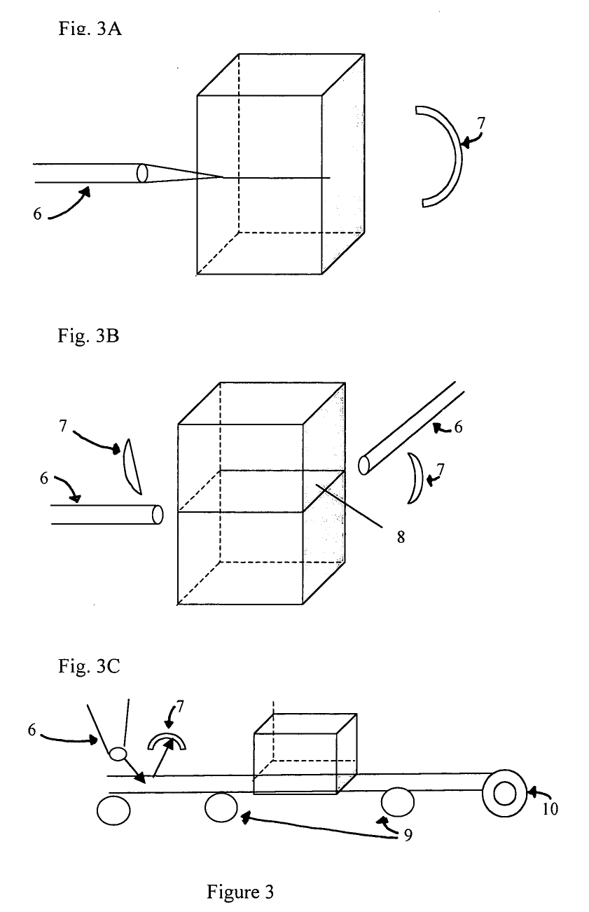

At the imaging layer within the length of the gel, a camera shines UV radiation (˜260 nm) onto the gel with different depth of focusing (FIG. 3A). At each focusing, we can obtain the passing of certain samples. We then move the UV light beam a slight step inward and focus it there. For example, with the design of 1,000×1,000 samples in loaded in fixed locations, we can do 1,000 focusing steps to obtain the light intensity for all the 1 million samples.

2. 2-Dimensional Image Reconstruction Using Software

Another way to get the trace images for each sample is through image reconstruction technology similar to that used in a typical CAT scan (FIG. 3B). Here, two laser beams from different angles (for example, placed perpendicular to each other) irradiate the gel at a fixed 2-dimensional imaging layer at the same time. The emission from those two different light sources is recorded at distinct time steps as is done in a regular gel-imaging device. A computer program can then be applied to calculate the light emission intensity within each point inside the surface (of course as reconstructed at a certain mesh density).

3. Printing of the Sample onto a Medium at Distinct Time Steps

On the bottom side of the gel-cube or capillary matrix, a thin layer of medium, such as paper, film, plastics, cellulose, or the like (henceforth simply referred to as paper) that is driven by a motor is placed at a proper distance (FIG. 3C). This paper is conductive, as the electrophoresis has to be ongoing with the presence of the paper. The paper is moving at a time-step of about 0.1 to 1 second for about each 0.5 to 4 cm move. The paper may move in one or two dimensions if it is wider (for example 2-10 prints in one dimension and hundreds or thousands of prints in the unwinding direction (several meters long rolled strip of material). The electric field can be turned off temporarily when paper is moving. As the paper stops moving, the DNA fragments with dye terminator coming out of the gel will print its content onto the paper. Because it is not always possible to keep all the samples running in synchrony, there are about 3-10 stops per peak (i.e. band for a given base). Thus, for a 1,000 base read length, there are about 3,000-10,000 paper prints is set. If a gel-run takes 100 minutes (6000 seconds) then a printing speed of about 0.5 to 2 frames/second is set. This speed is achievable with standard mechanics and electronics. The paper prints are then read by a standard or adjusted array scanners (which can be, for example, charge-coupled diode (CCD) based, a photon detector, an electron detector, or the like) to generate time point images of the entire sequencing matrix. The time-image for each sample can be reconstructed from those frames using computer software well known to those in the art.

In one embodiment two or more glass, plastic, polymer, or metal plates, or the like, may be used to deposit exiting DNA or polynucleotide. The polynucleotide can be genomic DNA, cDNA, RNA, ESTs, oligonucleotides, a derived polyncleotide, such as aptamers, a synthetic polynucleotide, or the like. The nucleotide can comprise at least one base, such as, but mot limited to, adenine, guanine, cytosine, thymine, uracil, a chemical derivative, such as having a methyl group attached, a metabolic precursor, such as orotate, or the like. The nucleotide can be in the deoxy-form or a dideoxy-form, or an equivalent thereof. Many such nucleotides are known in the art. The plate may act as an electrode. After DNA is deposited enough time on one plate, that plate is moved to one side and second plate is inserted in the collecting position. The first plate may be read and cleaned during collection time on the other one or more plates. The plates may be illuminated from above or below or from a correct angle or horizontally through material to produce total internal reflection (TIRF). TIRF illumination may be achieved by sweeping laser back and forth or by defusing it. TIRF may be used to perform imaging by using a single plate. The old dye molecules would photo-bleach. In this case the plate may have to be cleaned only from time to time. During such cleaning steps the electric field may be reduced in strength or turned off.

For this or all other imaging approaches a CCD array may be used. CCDs may have about one to four million pixels or may be produced with ten million or more pixels. Each separation unit (gel section, or capillary channels) may be monitored with one or multiple pixels using proper objectives and other optics. Thus even over one million separation channels may be imaged or monitored in parallel obtaining a from between a few to several images per frames per second. Because for each of about 200-2000 DNA bands it would take about 1-10 or more seconds to move it through the system, 10-100 measurements can be obtained for each band to provide optimal differentiation of consecutive bands. Four-color discrimination may be obtained by using a color camera (thereby reducing the number of pixel available for each color), or by using four specific filters and black and white camera, reducing four fold number of measurements per unit time for each color. Multiple (for example, between two and four) CCDs may be used in parallel if the collected light is split.

4. Splitting Capillary Matrix into Aligned Capillaries

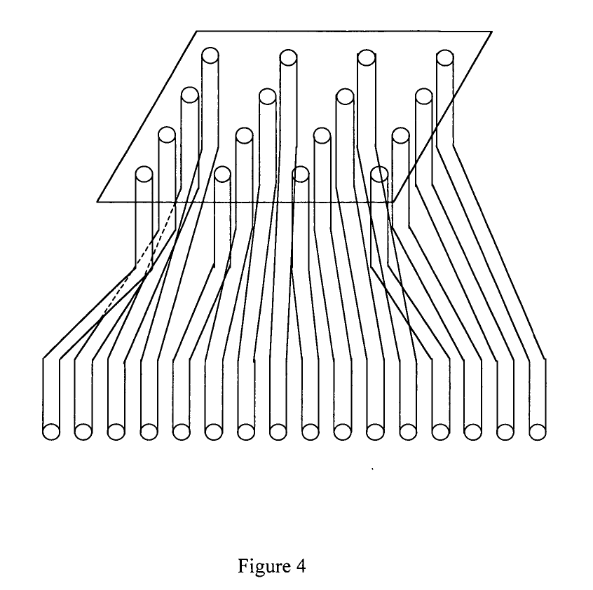

When DNA samples are separated within each fiber, there are a number of options for obtaining the trace image. For example, the flexibility of the fibers to gradually un-bundle them can be used (FIG. 4). The 3-dimensional fiber bundle can be gradually split in serial steps, until a 2-dimensional fiber bundle is created, where all the fibers in the original fiber matrix are aligned next to each other in straight line (a 1-dimensional fiber array, 1-D array for short). The laser scanner is applied only to those 1-D arrays of aligned fibers (FIG. 4). The un-bundling process may be done at different level to create smaller 2-D groups that may simplify illumination of imaging. In this way, more traditional type of scanner will be sufficient to obtain the sequence traces. No imaging reconstruction is needed.

5. Applying a Reflection Surface or Cutting the Fiber with Tilted Angle

The simplest illumination and imaging of gel-cube or capillary matrix is by exposure of the end surface with light and collecting the light emitted by dye molecules using properly positioned optics and detectors that do not interfere with the electrophoretic field. The end segment or surface material in the separation channels may incorporate components that may prevent penetration of light inside of the separation channels to excite other bands and photo-bleach dyes before they get in focus for detection.

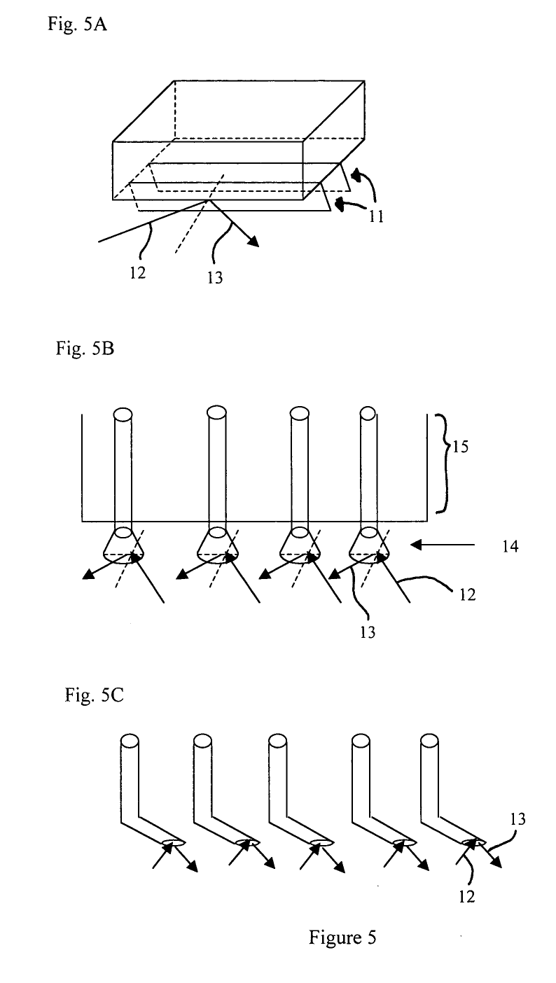

For the gel-cube, a set of plates with flat surface that provide light reflection at the bottom part where the DNA bands exit from the gel are used (FIG. 5A). The lamp or laser light, at the correct angle, is shone on the exterior surface of the gel-cube to excite only the dye terminators in the exiting bands and is reflected back without exposing and potentially photo-bleaching DNA bands that are retarded in the gel matrix and still outside of the detection area. For the fiber matrix, a layer of tilted tubes that are half-open can be used whereas the other half is coated with light reflection material (FIG. 5B).

In the alternative, simply by bending fibers and then cutting them with a fixed angle (creating a cut that is at 90 degrees relative to the longer unbent part of the fibers) the correct or proper angle for the light reflection can be created (FIG. 5C). The fibers may be grouped in 2, 4, or more groups and bent at different angles or positioned at different spacing for illumination of smaller areas using multiple light sources. Internal fiber surface at their ends can be coated with some reflective compound. Light can be collected by photo-multiplying tubes or a CCD chip having a capture speed of about 10 frames per second. A flow of liquid within the structure may be used to reduce heat and bring DNA bands to the focus area.

6. In Situ Illumination

The light transmission properties of fibers that are used for separation of the polynucleotides may be combined with other fiber optic cables or fibers to bring light; light may be passed from top to bottom of separation matrix walls without illuminating the separation medium and polynucleotide inside of capillaries. The light is reflected under different angles at the end of capillaries by properties of an end-added compound to illuminate dye molecules that are linked to the polynucleotide or DNA that is exiting the capillaries or that remains inside but close to the end of capillaries.

In a different implementation, a plate or layer of light-producing semiconductor or other material (spontaneously or when exposed to electricity, such a semiconductor quantum dots or the like) may be added to the end of gel cube or capillary matrix (extending capillaries or matching wholes in the added plate with capillaries). Light may be directed horizontally toward the holes to excite the exiting labeled DNA.

III. Sample Preparation

1. Amplification and Preparation of DNA Samples by Universal Primers

This approach does not require but can benefit from the sequence of an example/reference genome, and thus it provides efficient, highly parallel sample preparation for de novo sequencing of new genomes or their segments or cDNA libraries. In a typical application of sequencing the complete genome of a species, a long clone, a mixture of short clones, or a mixture of selected segments, comprises the following steps:

1) Preparing the random genomic segments of about 1,000 bp in size. The size selection can be made after the genomic sequences are broken down to pieces using DNAse or restriction enzymes or mechanical fragmentation. Another embodiment is to prepare library of targeted segments for example by use of specific restriction enzymes that may be combined with end matching adapters.

An especially efficient way of making a targeted library is use of mixtures of sequence specific primers that may be tagged with biotin or otherwise for isolation of synthesized DNA segments. These primers can be selected for isolating and sequencing genes or control regions of interests, or properly spaced to get more even sequence coverage of genomic DNA. One way to beneficially use mixtures of primers is to create smaller fractions of genome that can be analyzed in different runs or on different units in arrays of gel-cubes or capillary matrices. Genomic regions can be grouped by various criteria including guanidine-cytosine (GC) content to allow application of different DNA preparation and sequencing conditions. The primers can have a designed adapter tail with universal primer and restriction enzyme recognition sites. The primer pools can be used in a single or multiple extension steps providing no amplification or linear amplification. The pools may also contain pair of primers for exponential amplification. For some applications the length of segments produce may vary in a broad range from about 500 to about 50,000 bases. The produced fragments may be used directly or subjected to further fragmentation as one mix or after allocating in small portions that contain only a fraction of generated DNA molecules to obtain mapping information as described in the next paragraph.

Primers can be synthesized using methods well known to those in the art. Primers can have random and unknown polynucleotide sequence or can be specifically synthesized having a known polynucleotide sequence. Polynucleotides having random and/or unknown sequence are useful in that they can hybridize and bind to DNA fragments from many regions of a genome thereby enabling possible further increase in amplification copy number of a sequence of interest.

An embodiment of sample preparation can incorporate a two level fragmentation method previously invented by Radoje Drmanac and is herein described briefly. This method provides mapping information for assembling chromosomal haplotypes and alternatively spliced mRNAs for any random fragmentation, single molecule analysis methods. In this method, sample DNA is first fragmented in longer segments of about 5 to 10 to 100 to 500 kb fragments. By proper dilution a small subset of these fragments are at random placed in discreet wells of multi-well plates or similar accessories. For example a plate with 96 or 284 or 1536 wells can be used for these fragment subsets. The subsets can contain a few to 10, 10 to 20, or more fragments (including about 100 to about 1000 or more fragments). The fragment subset complexity is determined by the capacity of individual sequencing matrices and by statistics. The goal is to minimize cases where two overlapping fragments from the same region of chromosome or the two mRNA molecules transcribed from the same gene are placed in the same subset, e.g. the same plate well. In this way prepared groups of long fragments are then further cut to the final fragment size of about 200 bases to about 2000 bases. All short fragments from one well will be further processed in one sequencing matrix or in one section of larger continuous matrix. The above-described array of matrices or gel-cubes is very appropriate for parallel analysis of these groups of fragments. In the assembly of long sequences the algorithm will use the critical information that short fragments belong to a limited number of longer continuous segments each representing a discreet portion of one chromosome or one mRNA molecule.

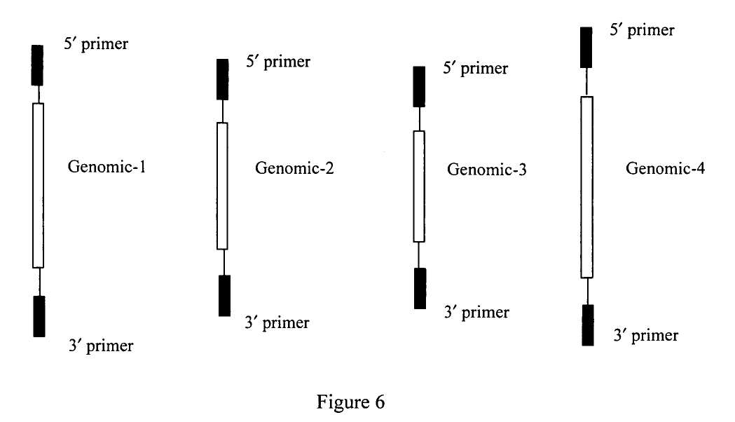

2) Connect each of those fragments to a universal primer-pair site of about 20-30 bp in size by ligating corresponding adapters to double stranded or single stranded DNA (FIG. 6). Usually adapters are prepared with several degenerated positions such as:

BBBBBBBBB

BBBBBBBBBNNNNNNN.

This provides all possible end sequences to capture all possible ends of sample DNA fragments. Target DNA molecules may be extended at 3′ end with about 10-50 As (or one or any of the other three bases) to use with an adapter with complementary tail (six or more Ts in this example). Adapters (depicted by Bs) have length in the range of about 10-100 bases to accommodate one or more priming and/or restriction or other sites. Adapters may be designed with or without addition of other connecting oligonucleotides to generate single-stranded circular molecules of target DNA fragments with a common synthetic segment with priming site for rolling circle amplification, and other optional sequence segments.

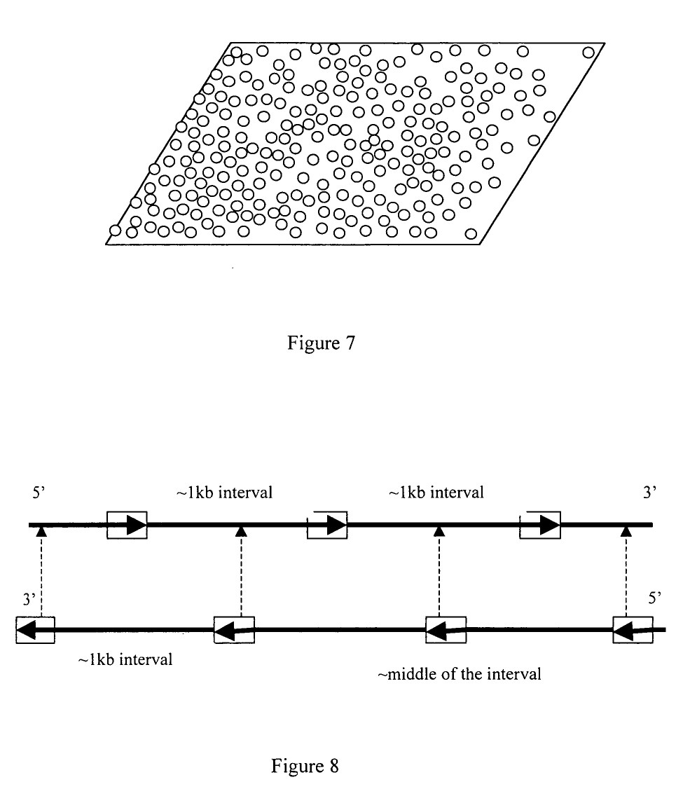

3) Apply the sample into a gel surface where the genomic segments are evenly spread in the surface with only single copies at individual locations (FIG. 7).

In another embodiment, the prepared DNA fragments can be diluted and loaded in various microstructures to obtain a maximal population of individual structures (wells, holes, or channels) occupied with single molecule of a DNA fragment. Loading may be adjusted to have more double fragments than no fragments because some fragments may not amplify, thus producing single amplified fragment as needed. An example of such structure can be a slice of a bundle of micro-tubes or fibers that provides thousands to hundreds of thousands or millions of discrete individual wholes (or wells if temporarily or permanently closed at one end with a solid or porous material). The structures can be loaded with DNA in a buffer or buffer and gel or other medium. Another example is a plate (glass, silica, plastic, polymer, metal, or other materials) with etched-through holes that may have for different designs a few microns diameter with about 5-10 microns center to center, or large diameters up to about 30-100 microns.

In another embodiment, circular target molecules are amplified by rolling circle method that produces long single-stranded DNA made of copies of the target fragment spaced with adapter sequence. In this case amplification can be done in a homogenous reaction at a dilution that minimizes interactions of produced single stranded molecules.

In another embodiment, diluted DNA fragments are loaded directly on top of gel-cube or in a gel layer on top of fiber bundle/matrix, or into gel loaded in the capillary fibers. This entry section of gel of fiber bundle is subjected to temperature control including temperature cycles if needed, depending on the type of amplification reaction used.

4) Amplification of single molecule DNA fragments using PCR or any other methods that provide necessary yield and accuracy, where each segment is amplified to 10³-10⁸ copies, preferably 10⁵ to 10⁷ copies. Amplification may be done on top of separation medium or in separate devices. High fidelity polymerases may be used to minimize generating errors during the extensive amplification.

Usual DNA concentration obtained by PCR is about 10¹⁰ to 10¹¹ molecules/mm³. Thus, a well having dimensions of about 10×10×1000 microns can have between about 10⁶ to 10⁷ molecules. Thus, sufficient amount of DNA (preferably >10⁵) is provided even in very small wells, for example about 3×3×300 microns in dimension. The amplification products are localized (e.g., at most of the locations all amplified fragments have the same origin from a single original molecule) because of the semi-solid nature of the gel or walls of the used microstructures. One amplification primer may be attached to the walls of structure or beads loaded in the structure to simplify cleaning. Primers may have tail segment with restriction sites or incorporated uracil. One primer may be phosphorylated to allow lambda exonuclease digestion of one strand and production of ssDNA for the next step. Two runs amplification can be done using the same or nested primers. Fragments may be removed from the support if attached primers are used using restriction cutting or uracil cutting. If beads are used they may stay in the structure during the next step after DNA is released from them, and in one embodiment captured by primer oligonucleotides attached to a second bead set loaded into structures after the first step is completed. By combining an exonuclease cut, cleaning and removal of exonuclease with a subsequent cut from the support, single stranded DNA with 5′ phosphate may be produced for use in the next step.

5). Dye-terminated linear amplification step (similar to cycle sequencing reactions) or one-time extension without cycling, where the dye terminators are mixed with normal nucleotides at fixed ratios, can be used. As a result, the dye-terminators are incorporated into the newly synthesized DNAs with varying sizes. An alternative is to use dye labeled primer or any other labeling or termination or base specific fragmentation chemistry.

In one embodiment, small beads (diameter from about 0.1 micron to about 30 microns) with an attached universal sequencing primer are loaded in the structure (one or more per unit structure) and single stranded DNA molecules are annealed to primer molecules. In the case that amplification was done with one bound primer, beads may be loaded before releasing DNA from the structure walls. The releasing agent may be loaded together with beads. Sample can be cleaned of all unbound components before adding buffer with dye terminators and a polymerizer. After terminating the extension reaction, 5′exonuclease or denaturating conditions may be used to remove original template strands. The reaction may be cleaned by flow-through. The result will be clean single stranded fragments terminated at different positions (and labeled according to the end base) still attached to beads. DNA fragments may be released from the beads in the structure or after beads are transferred into sequencing matrix.

In another embodiment, DNA is amplified using beads with one attached primer (either in the microstructures or in emulsion that separates beads), or an amplification primer attached to the walls of microstructures. After removing non attached DNA strand and easy cleaning (e.g. replacing buffer) sequencing primer is hybridized to templates on beads and dye-terminators incorporated. If beads are separated in microstructures, or template DNA anchored to the microstructure walls, cycle sequencing method may be used to produce free labeled DNA fragments ready for loading into separation medium. If all beads are processed together in one homogeneous dye-terminator incorporation reaction then individual beads are spread onto sequencing matrix (e.g. one per capillary), and labeled fragments are separated by denaturing, and loaded into separation medium. A bead of about 10 microns in diameter may hold over one million template molecules thereby providing hundreds of labeled molecules for each base. For single-bead load approach, smaller or larger beads may be used in the range of about 1 to 100 microns depending on detection sensitivity and sequencing capillary size.

If the rolling circle method is used for amplification, a simplest approached is to dilute amplification reaction into sequencing reaction with sequencing primer and dye-terminators to produce labeled sequencing fragments that will hold together on their long chain of templates. Dilution may provide replacement for purification but it will still provide enough templates for loading a matrix of, for example, 10 micron capillaries. Other modification may be used to provide good yield and to keep chain to some extend coiled instead of extended. A stopper or capture oligonucleotide can be used in solution or micro beads that is identical to a portion of the incorporated adapter separated by between about 3 to 30 bases from the priming site. This oligonucleotide is complementary to the single-stranded DNA (ssDNA) so produced and can provide a stop for the complementary strand produced by sequencing primer (if it is not stopped by dye-terminators) and preserve portion of ssDNA. Also, if attached on beads it provides a capture oligonucleotide for the produced ssDNA to keep them localized to the bead surface. Various enzymatic or chemical treatments may be preformed after amplification or after dye-terminator or similar stoppers incorporation to degradation or block or deactivate dNTPs or primers or enzymes or other reaction compounds. Rolling circle amplification or dye-terminator incorporation may be performed after loading input sample into sequencing matrix.

Individual randomly (including hairpin-directed) coiled rolls are loaded into the gel surface or capillary channels where labeled fragments are denatured for separation. In a between about 100-1000 μl amplification reaction having individual circles occupying a 3-5 micron cube (having a low chance of interacting; one million copies of a 1 kb polynucleotide) there are hundreds of millions of amplification circles. By diluting this reaction by between about 10-1000 fold the density of individual templates for sequencing is sufficient for loading (by spreading or spraying) apportioned amount of approximately 0.01 to 1 nl per sequencing channel.

6a). Sequencing in a gel-cube where 2-dimensional images are collected and decomposed using computer algorithms; loading from the externally prepared samples is done through surface contact capillary forces or active electrical or pressure/vacuum forces.

6b). Sequencing in capillary or fiber matrices that starts immediately beneath a reaction surface. Images from the fiber matrices can be obtained by de-bundling the fiber into a single linear array of the fiber gradually or using other described imaging and detection methods. A capillary/fiber bundle slice used for highly parallel DNA fragment amplification and Sanger or other sequencing reactions allows efficient simultaneous loading of samples into sequencing capillaries/fibers filled with a separation medium. The bundle type used for sample preparation slice may have ticker walls and smaller internal diameter of capillary channels in comparison to sequencing bundle. In addition, contact surfaces can be coated with hydrophobic material to prevent horizontal flow of water-based buffers. By putting the slice in contact with top surface of sequencing matrix a great majority of samples will be positioned above single sequencing capillary. DNA molecules or beads are then transferred by capillary, gravitational, electrical or pressure forces. Separation material loaded in the capillaries may be less or more dense at the beginning of the capillaries to allow better transfer, and collecting DNA molecules in a narrow band to provide sharp single base resolution separation. Due to random nature of loading single DNA fragment molecules into structures, a large fraction of capillaries may have no sample or multiple samples. This potential inefficiency is offset by very large number of parallel sequencing channels.

2. Using Microarray or Bead Array to Capture Genomic Segments

An alternative to in situ amplification is to use either a microarray (solid surface, membrane, micro-wells, or the like) or bead array to capture genomic segments.

1) Oligomer Set Selection

Specific oligomers are pre-spotted or in situ synthesized onto the surface of a microarray or a pool of beads. The length of oligomers should be determined by the genome in hand. For human genome, one can pick 30-60mers, with similar melting temperature. The oligomers should be picked in such a way of at least 2×-coverage of the genome. The oligomers are designed to be a tile-coverage of both strands of a reference genomic sequence, thus 1× is the forward strand, and 1× is from the reverse strand (FIG. 8). In addition, the primers picked from the forward and reverse strand may or may not be overlapping with each other. This is to help identify SNP (simple nucleotide polymorphism, including substitutions and simple deletion/insertions). The genomic fragments should be relatively similar in length, and be purified from a donor, fragmented, then segments of about between 1000 to 3000 bp are selected. Primers may be selected to be maximally distinct to minimize mixed DNA fragments representing gene family members in the same sequencing reaction.

2) Capturing Specific Genomic Segments with Sequence-Specific Hybridization

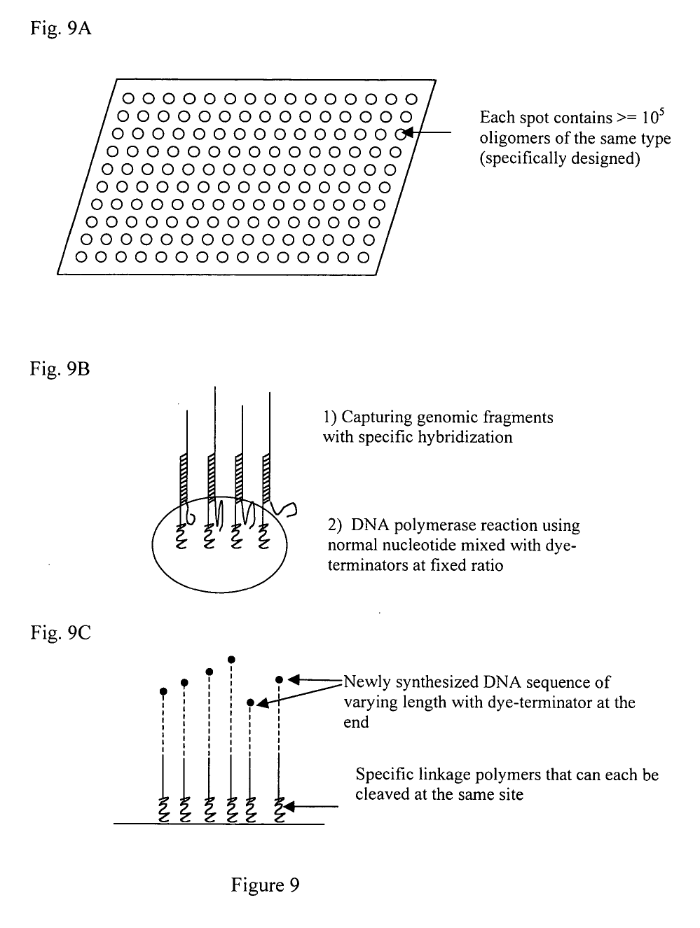

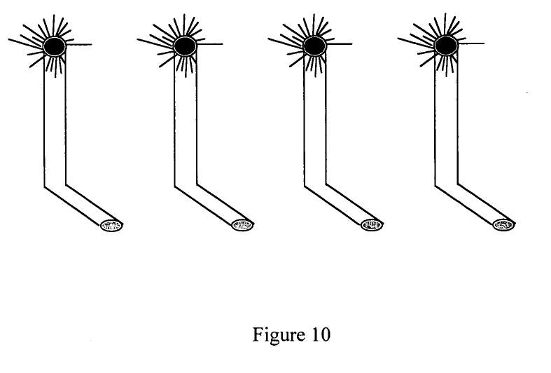

In this step, genomic fragments of similar length are prepared first and applied to the primer microarray surface (FIG. 9A). Reaction conditions are controlled such that hybridization will occur. A hybridization step at a high temperature can be performed in order to avoid unspecific bindings from segments within the genome that is non complimentary to the oligomer being used. At each spot on the microarray, a population of genomic segments that contain the complimentary sequence to the spotted oligomer are captured through hybridization (FIG. 9B). Whole genome amplification can be performed to produce enough sequencing templates.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2nd ed., vol. 1-3, Cold Spring Harbor Press, Plainview N.Y.; specifically see volume 2, chapter 9.

3) In Situ Extension of Oligomers and the Incorporation of Dye-Terminators

An in situ linear cycling polymerase reaction is now performed to extend the oligomers attached to the solid surface, using the hybridized genomic fragments as a template (FIG. 9B). The deoxy-nucleotides that are added to the solution is a mixture of both normal and dye-terminated at a fixed ratio. As the DNA polymerase extends the oligomer, it will stop if one of the following happens:
The end of the genomic fragment is reached. The newly synthesized DNA has no dye-terminator attached.
The end of the genomic fragment is not reached, but a dye-terminator is incorporated at the end.

The mixture of normal deoxy-nucleotides and dye-terminated nucleotides are in such a ratio that the majority of oligomers can be extended to a length having the dye-terminator at the end.

Those sequences would all have the same 3′ end group, as they will be terminated as they contact the end of the spotted DNA probe on the microarray surface.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Aproach, M. Schena, editor (1999) Oxford University Press, London, hereby expressly incorporated by reference in its entirety.

4) Dehybridization and Washing Away Genomic Fragments

The temperature is then increased and reagents are added to dehybridize the genomic fragments from the newly synthesized fixed DNA sequence. After the dehybridization, the genomic segments are washed away from the array or the microbeads.

What is left is the microarray with oligomers extended with genomic segments (to be sequenced) (FIG. 9C). On each spot, sequences have the same 5′-end, namely the oligomer used as anchor. But the length of the extended sequences should vary greatly, with from between 1 to about 1,000 bp in length. The 3′-ends of those sequences are of two types: those that end with normal nucleotides and those end with the dye-terminators. We will focus only on the ones with dye-terminators at the end, as the others will not generate color signal when excited by laser beam. If we start with ˜2*10**6 molecules per spot in the beginning, we expect about 50% will be with dye-terminators at the end, e.g. about 10⁶ molecules. If we assume an evenly distribution of length among those 10⁶ molecules, for a length of 1,000 bp, we will have 1,000 molecules for each distinct length between 1 and 1000. Of course, the molecular density will not be evenly distributed, at each specific length, the number of molecules will be in the range of about 100-10,000.

5) Optional: Removing Oligomers Without Dye-Terminators at the End

Because some of the hybridized DNA with the oligomer may be short on the sequence post the hybridization site, the cycle deoxynucleotide extension on the oligomer may be terminated without the incorporation of a dye terminator. Those extended oligomers form an exposed population and may interfere with the electrophoresis. Exposed (naked) DNAs can be removed using an enzyme, for example 3′ exonuclease that is blocked by dye-terminators.

6) Releasing DNA from the Surface and Running Electrophoresis

Because the oligomers are anchored on the solid/membrane surface with uniform anchors, an enzyme is added that will specifically release the DNA fragments from the surface (FIG. 9C). This release will be uniform on all spots as the same compound is used in anchoring.

The released molecules should be contained within the neighborhood of the spot. This can be done by contacting a gel-cube or a fiber-matrix with the microarray. The contact surface of the gel-cube or the microarray fiber tip comprises the releasing enzyme. A certain time is allowed for the reaction to complete. The objective is to capture the newly released DNAs either into the fiber channel, or into the gel-cube. In one scenario, tiny holes (wells) can be created on the surface of the gel-cube the size of the spot on the microarray. The tiny holes contain the solution with the releasing enzyme. The microarray is placed and fixed on top of the gel-cube together. The system is shaken slightly to let the solution within the tiny holes to mix with the spotted molecules.

7) Using Membrane Matrix and Beads as Alternatives to Solid Surface Microarrays

There are several variations to the technique outlined above. One is to use a microarray with membranes fixed instead of with solid flat surface. All the processes outlined above and herein would essentially apply to this scenario with no change, and so the detailed steps and not further described here.

Another alternative to using microarray with spotted oligomers is to use micro-scale beads with spotted oligomers. Assume that the oligomers are prefixed onto the bead surface before our experiment and that there is a well-mixed bead collection inside a tube or any container; for example, the is a 2×-coverage of oligmers for the genome with fixed gap length of about 1,000 bp. The reaction of oligomer extension is performed inside the tube. The end product would be each beads contains a mixture of DNAs of the same 5′-origin (as specified by the oligomer anchers). The other steps would be the same in extending the oligomers into genomic segments with the dye-terminated DNAs at the end, except now the reactions occur at the surface of the beads instead of the surface of the microarray. Assume that the genomic segments have been extended onto the oligomers on the bead.

The beads are applied onto a fiber matrix surface where each fiber may capture one or zero beads at its end (FIG. 10). By rotating the beads, their surfaces are turned such that each side of the bead get some exposure inward to the capillary. If there is a solution within the capillary that contains the enzyme that can release the oligomers, then electrophoresis is performed as disclosed above.

### DETAILED DESCRIPTION OF THE DRAWINGS

FIG. 1. Gel-cube and capillary fiber matrix. Gel may be separated by vertical mesh that guides the sample to move only in one direction. Capillary matrix can be fixed together at one end, and split in the other if needed.

FIG. 2. Gel-cube arrays or fiber arrays for temperature difference, application of different samples, or with different reaction specifications.

FIG. 3. Three different methods to read out the DNA sequence from the gel-cube or capillary matrix.

FIG. 4. Vertical fibers coming out from a cube-shaped apparatus is re-aligned into a I-dimensional linear array of fibers, where a scanner can scan it easily.

FIG. 5. Laser excitation and reflection at the exit surface of gel-cube or fiber matrix with reflecting surfaces in close contact with the gel-cube or fiber matrix. A: reflecting surface is composed of tilted metal plates; B: reflecting surface composed of half cylinders; C: bend the capillary at the end so that the cut edge has an angle to reflect laser light.

FIG. 6. Genomic fragments of DNA with varying length and adapters for universal primers attached at each end.

FIG. 7. Gel surface with random spread single copies of genomc DNA, to be in situ amplified by PCR reaction, also, to be linearly expanded to generate samples containing dye-terminator DNA fragments of varying length.

FIG. 8: Selection of oligomers with a 2× coverage of the genome. The first set for the top strand is selected such that: 1) the intervals between the primers is ˜1 kb; 2) the oligomers have no close homologs within the genome. The second set for the reverse strand is selected in addition to 1) and 2), but also: 3) close to the middle points between two neighboring oligomers in the forward strand.

FIG. 9: The generation of dye-terminator ended sequences ready for electrophoresis using a microarray of specifically designed oligomers.

FIG. 10. Capillary array with beads on some or all capillaries. The beads are loaded with DNA segments. Enzymes within the capillary can release the DNA fragments from the bead so that a gel electrophoresis can be run.

### LIST OF REFERENCE NUMERALS

1. Optional grid where DNA or polynucleotide is placed
2. Optional film or films that separates the gel into layers or grids
3. Solid case
4. Physical or material separation between component subunits
5. Gel-cube or fiber matrix
6. Laser light beam focused on a layer of the gel-cube or fiber matrix
7. Emitted light collector or detector
8. Scanned surface
9. Motor to drive paper or recording medium
10. Paper roll or recording medium storage means
11. Titled or angled reflective surface or medium
12. Photon input
13. Photon reflected
14. Tilted half circle or prism structure

The invention will be more readily understood by reference to the following examples, which are included merely for purposes of illustration of certain aspects and embodiments of the present invention and not as limitations.

### EXAMPLES

Sequencing the Complete Human Genome in One Run

The human genome has about 3 billion base pairs (bp) of nucleotide sequences. Sequencing the complete genome in a single step or a few integrated steps is an objective that many institutions and investigators are targeting. Here we describe processes, methods, and systems for achieving that objective. The basic idea is using traditional dye-termination sequencing, but employing new techniques to massively parallelize the process as described above.

A complete human genomic sequence (reference genome A) and the complete genome of another individual (test genome B) are sequenced to find the differences of B as compared to A. Because A and B genomes are both from human, the differences are mostly SNPs (single nucleotide polymorphisms). Genome B may be heterogeneous, in the sense it is actually composed of two complete genomes, B1 and B2, where each copy is from one of the parents.

1. 10× Coverage Genome Sequencing with Random Amplification

Assuming a 3 billion base pair (bp) genome, for a typical sequence read of ˜1,000 bp it take ˜3 million reads to complete the genome sequence. Given the random nature in sampling for genomic segments as given in section III, about 10× coverage is needed in order to obtain a genomic sequence with >95-99% completeness. A 10× coverage means we would need 30 million reads. In a gel-cube or capillary matrix, this 30 million reads are obtained with exemplary dimensions: 3 cm(width)*10 cm(length)*20 cm(height), if the average density of randomly placed DNA samples is about 10 mm apart. The top surface area is 3 cm×10 cm where all the reactions, except electrophoresis, occur. With an increased density of the DNA samples, a gel-cube or capillary matrix with smaller size is used to achieve the same objective. The volume of 30 million of nano amplification reactions, each about 0.1 nl (10 micron×10 micron×1000 micron reaction chamber unit) to 1 nl, is 3-30 ml. With an approximate cost of one cent per ml the cost of amplification process may be $30-$300 or less, thus allowing sequencing of a whole human genome for $1,000.

2. 2× Coverage Sequencing of a Heterozygote Genome with Specific Designed Primers from a Given Genome

Two oligomer sets from known Genome A (or Sequence A) are designed. The first set is selected in the forward orientation, 5′->3′, and the second set is from the complement sequence from the same genome, Sequence A^{c}, also 5′->3′. The oligomers are between about 500 bp-1000 bp apart from each other, depending on read length and on quality requirement. Oligomers are selected such that they will be of varying length, but have a relatively homogeneous melting temperature. The typical length of oligomers is 20 bp-60 bp, and more likely 30 bp-40 bp. When oligomers are selected, those that have low homology to other sequences within the genome are preferred. This is achievable since relatively long oligomers are used (up to 60 bp). Let the oligomer set picked for Sequence A (A-O set) and the oligomer set picked from Sequence A^{c} set the A^{c}-O set. The A^{c}-O should be the complimentary sequence within the middle regions of the Sequence A as partitioned by the A-O set, and vise versa. In this way the best coverage of the genome and the best likelihood of detecting and resolving all the SNPs are obtained.

A microarray with the specific oligomers (A-O set and A^{c}-O set) fixed to each spot is provided having a 2×-coverage of the genome sequencing with 1× cover for one orientation of the genome, and the other 1× the reverse orientation. With 500-1,000 bp read length for each spot, 6-12 million reads (6,000,000×1,000 bp is 2× of the genome) are performed. Thus, using the size of spots mentioned above (10 micron×10 micron), a 2 cm(width)*3 cm(length) microarray is used having sufficient number of oligomers. Such a microarray is fabricated from in situ synthesization as the case of Affymetrix chips or each oligomer can be synthesized first and spotted onto the microarray.

This microarray first captures the DNA fragments from the heterozygote genomic segments of a person. The hybridization occurs at a relatively high temperature that is slightly below the melting temperature of the oligomer sets. Alternatively, the hybridization conditions are adjusted by altering the stringency ([Na⁺]) and pH. For 20-30 mer primers the temperature is between 40° and 70° C. This hybridization with high temperature minimizes impurities associated with imperfect hybridization. After the hybridization, the remaining DNA fragments that are not bound to the chip are washed away using standard buffers used in array hybridizations (see, for example, Sambrook, J. et al. (1989) Molecular Cloning: A Laboratory Manual, 2nd ed., vols. 1-3, Cold Spring Harbor Press, Plainview N.Y.;). Now the temperature is returned to normal (20°-50° C.).

A dye-terminator incorporation extension step follows. In this step cycle extension of the spotted oligomers and dye-terminator incorporation on the microarray is performed. The microarray is then put on top of the gel-cube or fiber matrix to perform enzymatic release of DNA fragments and then electrophoresis.

Alternatively. the microarray is replaced by a bead population of 6-12 million unique beads. Each bead contains a specific oligomer designed from the known genome. The beads are mixed together within a tube, and the reactions of hybridization, cyclic extension with dye-terminator incorporation then occurs within the tube. After that, the beads mixture are applied to the surface of the gel-cube or the fiber matrix, where the tip of each grid within the cube or the fiber opening will serve to capture one bead per spot (FIG. 9).

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

We claim:

1. A reaction substrate having a plurality of surfaces comprising a composition suitable for sequencing polynucleotides, re-sequencing polynucleotides, genotyping, and SNP discovery, the substrate further comprising a plurality of primers anchored to the substrate and wherein each primer sequence is complementary to a specific polynucleotide sequence in a polynucleotide or genome of interest and wherein the primer further comprises a releasable anchor fragment, wherein the anchor fragment is released using means selected from the group consisting of heat and by chemical reagents, such as, but not limited to, enzymes and catalysts, wherein the released polynucleotide is passed through a medium selected from the group consisting of a microfiber, a mesh, and a gel-cube, and wherein the reaction substrate is selected from the group consisting of a microarray, a micromatrix, a microarray plate, a plurality of beads, and a micro-structure.

2. The reaction substrate of claim 1 wherein the primers are at a density selected from the group consisting of 1,000, 1,001-10,000, 10,001-100,000, 100,001-1,000,000, and 1,000,001-10,000,000 primers per substrate.

3. The reaction substrate of claim 1 wherein the primers are of length selected from the group consisting of between about 10-20 bp, about 21-30 bp, about 31-50 bp, about 50-100 bp, about 101-200 bp, and about 201-400 bp.

4. The reaction substrate of claim 1 wherein the primers are selected from the group consisting of random primers and primers having known polynucleotide sequence.

5. A method for sequencing DNA fragments using the reaction substrate of claim 1, the method comprising the steps of:
i) providing the reaction substrate of claim 1;
ii) providing DNA fragments of interest;
iii) hybridizing under stringent conditions DNA fragments that contain the complimentary sequence to the portion of the primer that is releasable;
iv) optionally removing DNA fragments having miss-matches to the primers resulting in the hybridized DNA fragments having greater purity, wherein removing the DNA fragments is performed using means selected from the group consisting of heat and physical means;
v) adding DNA polymerase, nucleotides, and dye-terminators to the reaction substrate;
vi) incubating the DNA polymerase, nucleotides, and dye-terminators with the primers and hybridized DNA fragments to extend the primers complementary to the DNA fragments using the DNA fragments as a template in a sequencing reaction wherein the primers are extended to form a strand and whereby the dye-terminators are randomly incorporated into certain portions of primers to create an anchored DNA;
vii) decoupling the hybridized DNA fragments from the anchored strand using means selected from the group consisting of heat and physical means, the means being selected from the group consisting of low stringency wash at 50° C. and a high stringency wash at 42° C.;
viii) washing the substrate thereby removing the decoupled DNA;
ix) releasing the anchored DNA from the surface of the substrate using enzymic or physical means; and
x) passing the released DNA through a medium; sequencing the DNA in the medium using three-dimensional imaging, the medium comprising three-dimensional microstructures selected from the group consisting of bundles of capillary fibers, a gel-cube, and a mesh.

6. A process for sequencing DNA comprising the steps of:
i) parallelized preparing of DNA sequencing reactions using DNA sequencing samples and a detectable composition wherein the detectable composition corresponds to specific DNA bases and is selected from the group consisting of at least three dyes, labels, and tags;
ii) parallelized loading of prepared DNA sequencing reactions on a separation medium wherein the loading is performed using a force selected from the group consisting of gravitational, capillary, and electric forces;
iii) running electrophoretic separation of DNA fragments;
iv) illuminating the detectable composition in time points for each separation element at a location proximal to the end of separation medium;
v) detecting the detectable composition; and
iv) determining the base sequence from the time profile of intensities of the detectable composition, thereby sequencing the DNA sequencing samples.

7. The DNA sequencing process of claim 6 wherein the number of DNA sequencing samples are selected from the group consisting of more than 1000, 10,000, 100,000, and 1,000,000 DNA sequencing samples.

8. The DNA sequencing process of claim 6 wherein the detectable composition is selected from the group consisting of target sequence specific primers attached to beads and target sequence specific primers attached to an array support.

9. The DNA sequencing process of claim 6 wherein the separation medium is selected from the group consisting of a separation matrix with corresponding capacity, a gel cube, a mesh, and a matrix of sequencing capillaries.

10. A process for sequencing DNA comprising the steps of:
i) parallelized DNA amplification from single DNA molecules using universal primers in a matrix having a corresponding number of microstructures loaded by capillary forces;
ii) parallelized sequencing reaction using the amplified DNA and four dye terminators in the same matrix of microstrucutres that may be loaded with beads with sequencing primer resulting in sequencing samples;
iii) parallelized loading of sequencing samples from matrix of microstructure to matrix of sequencing capillaries by capillary or electric forces;
iv) runing electrophoretic separation of sequencing samples;
v) illuminating and detecting four flourophores in time points at specific location close to the end, inside or outside of capillaries;
iv) detecting four flourophores;
vii) determining the base sequence from the time profile of intensities of four colors in the sequencing samples, thereby sequencing the single DNA molecules

11. The DNA sequencing process of claim 10 wherein the number of single DNA molecules is selected from the group consisting of more than 1000, 10,000, 100,000, and 1,000,000 single DNA molecules.
